(19) United States
(12) Patent Application Publication
(10) Pub. No.: US 2007/0218141 A1
(43) Pub. Date: Sep. 20, 2007

(54) SIMVASTATIN COMPOSITIONS

(76) Inventors: Ashis Kumar Mehta, Hyderabad (IN); Gurvinder Singh, New Delhi (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/685,411

(22) Filed: Mar. 13, 2007

Related U.S. Application Data
(60) Provisional application No. 60/807,572, filed on Jul. 17, 2006.

(30) Foreign Application Priority Data
Mar. 15, 2006 (IN) 467/CHE/2006

(51) Int. Cl. A61K 31/22; A61K 9/14

(52) U.S. Cl. 424/489; 514/548

(57) ABSTRACT

The present invention relates to pharmaceutical compositions comprising simvastatin or pharmaceutically acceptable salts, solvates, enantiomers or mixtures thereof, processes for preparing the same and methods of use and treatment.

### INTRODUCTION TO THE INVENTION

The present invention relates to pharmaceutical compositions comprising simvastatin or pharmaceutically acceptable salts, solvates, enantiomers, polymorphs or mixtures thereof, processes for preparing the same and methods of use and treatment.

3-hydroxy-3-methylglutaryl-coenzyme A (“HMG-CoA”) reductase catalyzes the conversion of HMG-CoA to mevalonate, which is an early and rate-limiting step in the biosynthesis of cholesterol. Inhibitors of HMG Co-A, frequently called “statins,” are useful as cholesterol-lowering agents.

Simvastatin (Formula I), has the chemical name 2,2-dimethyl-1,2,3,7,8,8a-hexahydro-3,7-dimethyl-8-[2-(tetrahydro-4-hydroxy-6-oxo-2H-pyran-2-yl)-ethyl]-naphthalenyl ester, [1S-[1α,3α,7β,8β(2S*,4S*)-8aβ]] butanoic acid. It is commercially available in the form of oral conventional tablets of 5, 10, 20, 40 and 80 mg strength under the brand name ZOCOR™ and is manufactured by Merck.

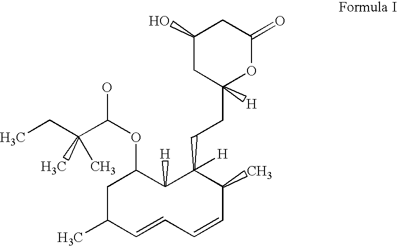

Particle size or particle size distribution of the active ingredient plays an important role in selection of dosage form, processibility of dosage form, dissolution and bioavailability. The rate of dissolution of poorly soluble drugs is a rate-limiting factor in its absorption by the body. A reduction in the particle size is expected to increase the dissolution rate of such compounds through an increase in the surface area of the solid phase that is in contact with the liquid medium, thereby resulting in an enhanced bioavailability of the compositions containing such compounds.

But the selection of a suitable particle size and a particle size distribution poses challenges to the formulators to design a formulation with all desired physico-chemical properties. It is generally not possible to predict the exact particle size and distribution that results in good physicochemical properties of active ingredient, good processibility during formulation and a composition that meets pharmacopoeial dissolution and bioavailability criteria, as different drugs show different dissolution characteristics with a reduction in the particle size.

U.S. Pat. No. 6,984,399 discloses pharmaceutical formulations containing lovastatin and simvastatin with a particle size D₉₀ between 15 and 100 μm and a specific particle surface area between 1 and 4 m²/g, which ensures rapid dissolution of the active substance from the pharmaceutical formulation

U.S. Pat. No. 6,696,086 discloses a process for the preparation of simvastatin or lovastatin in the form of crystals with D₉₀<40 μm.

U.S. Patent Application Publication No. 2004/0235935 discloses an oral pharmaceutical water-free (oily) suspension formulation comprising at least one statin with a particle size less than 500 μm. The statins are simvastatin, pravastatin and the like.

International Application Publication No. WO 03/078379 discloses a process for the preparation of amorphous HMG-CoA reductase inhibitor and hydrates, of desired particle sizes 1 to 150 μm.

The development of pharmaceutical compositions as described in the context of the present invention will permit a significant improvement in the field of clinical practice.

### SUMMARY OF THE INVENTION

The present invention relates to pharmaceutical compositions comprising simvastatin or pharmaceutically acceptable salts, solvates, enantiomers or mixtures thereof, processes for preparing the same and methods of use and treatment.

The present invention relates to solid pharmaceutical compositions prepared using simvastatin particles having a D₉₀ less than about 10 μm. The particles also have a specific surface area between about 4 m²/g and about 7 m²/g, or about 4.5 m²/g to about 6.5 m²/g.

### DETAILED DESCRIPTION OF THE INVENTION

The present invention relates to pharmaceutical compositions comprising simvastatin or pharmaceutically acceptable salts, solvates, enantiomers or mixtures thereof, processes for preparing the same and methods of use and treatment.

Surprisingly it has been found that pharmaceutical compositions prepared using simvastatin having a defined particle size distribution, wherein the simvastatin particles have D₉₀ less than about 10 μm, show rapid drug release but lesser values of Cₘₐₓ and AUC (and are bioequivalent with ZOCOR™ products) as compared to the pharmaceutical compositions comprising simvastatin particles having D₉₀ greater than about 10 μm. The D₉₀ is a value of the volume-based particle size distribution, such that 90% by volume of the particles have a size of this value or less.

The present invention relates to solid pharmaceutical compositions comprising simvastatin having a defined particle size distribution wherein the simvastatin particles have D₉₀ less than about 10 μm. The particles have a specific surface area between about 4 m²/g and about 7 m²/g.

In one embodiment of the invention, simvastatin with a defined particle size and distribution is provided. The simvastatin of the invention comprises of plurality of simvastatin particles having a particle size distribution D₉₀ of less than about 10 μm.

In another embodiment of the invention, the plurality of simvastatin particles has a specific surface area of about 4 m²/g to about 7 m²/g, or about 4.5 m²/g to about 6.5 m²/g.

Particle size reduction can be done by techniques known in the art, including but not limited to fluid energy milling or micronizing, ball milling, colloid milling, roller milling, hammer milling and the like.

The particles of simvastatin used can be single crystals, aggregates, agglomerates and any combinations thereof.

The particle size distribution of simvastatin of the present invention can be determined by techniques such as, for example, laser light diffraction, Coulter counter measurement, or microscopy. Other techniques for the measurement of particle size are also acceptable.

Further, the specific surface area of simvastatin of the present invention can be determined using standard techniques, such as a Brunauer Emmet and Teller (“BET”) surface area analysis method from the amount of gas that is adsorbed onto the particle surfaces, either under nitrogen adsorption or under vacuum.

The pharmaceutical compositions comprising simvastatin with or without pharmaceutically acceptable excipients may be formulated in the form of tablets or minitablets by processes such as dry granulation or wet granulation or direct blending. Resulting minitablets may further be filled into capsules.

In dry granulation, the granules may be prepared by sifting the active and excipients through the desired mesh size sieve and then mixing using a rapid mixer granulator, planetary mixer, mass mixer, ribbon mixer, fluid bed processor or any other suitable device. The blend can also be granulated by wet granulation. In wet granulation, the granulate can be dried using a tray drier, fluid bed drier, rotary cone vacuum drier and the like. The dried granulate particles are sieved and then mixed with lubricants and disintegrants and compressed into tablets or minitablets or filled into capsules.

Alternatively the manufacture of granules may be made by direct compression by using directly compressible excipients using a suitable device, such as a multi-station rotary machine to form compressed slugs or by roller compaction to form slugs, which are passed through a multimill, fluid energy mill, ball mill, colloid mill, roller mill, hammer mill and the like, equipped with a suitable screen. The milled slugs are then lubricated and compressed into tablets or minitablets or filled into capsules.

In context of the present invention, one or more pharmaceutically acceptable excipients may be used optionally to prepare the desired formulation. These pharmaceutically acceptable excipients may include but are not limited to diluents such as microcrystalline cellulose (MCC), silicified MCC (e.g. Prosolv™ HD 90), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders such as acacia, guar gum, dextrin, maltodextrin, methylcellulose, ethyl cellulose, hydroxyethyl cellulose, hydroxypropyl cellulose (e.g. KLUCEL®), hydroxypropyl methylcellulose (e.g. METHOCEL®), carboxymethylcellulose sodium, povidone (various grades of KOLLIDON®, PLASDONE®) starch and the like; disintegrants such as carboxymethyl cellulose sodium (e.g. Ac-Di-Sol®, Primellose®), crospovidone (e.g. Kollidon®, Polyplasdone®), povidone K-30, polacrilin potassium, starch, pregelatinized starch, sodium starch glycolate (e.g. Explotab®) and the like; antioxidants such as butylated hydroxy anisole (BHA), butylated hydroxy toluene (BHT), α-tocopherol and the like; surfactants including anionic surfactants such as chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, sodium lauryl sulfate (SLS) and sodium dodecyl sulfate (SDS); cationic surfactants such as cetylpyridinium chloride monohydrate and hexadecyltrimethylammonium bromide; nonionic surfactants such as N-decanoyl-N-methylglucamine, octyl a-D-glucopyranoside, n-Dodecyl b-D-maltoside (DDM), polyoxyethylene sorbitan esters like polysorbates and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like. Solvents that may be used in granulation or layering or coating are aqueous or alcoholic like ethanol, isopropanol or hydro-alcoholic like a mixture of water with alcohol in any ratio, or organic solvents like acetone, methylene chloride, dichloromethane and the like.

Pharmaceutical compositions of the present invention may further include excipients such as but not limited to pharmaceutically acceptable glidants, lubricants, opacifiers, colorants and other commonly used excipients.

The pharmaceutical compositions may also be formulated in the form of pellets (extruded or fluidized) or spheres or cores that are either encapsulated as capsules or compressed into tablets or minitablets; lyophilized powders and the like.

Core tablets or pellets can further be optionally film coated. The coating can be done by techniques known to one skilled in the art such as spray coating, dip coating, fluidized bed coating and the like.

The materials that can be used for the preparation of pellets or spheres or cores include but are not limited to: water-soluble materials such as sugar spheres, lactose and the like; and water-insoluble materials such as microcrystalline cellulose, silicon dioxide, calcium carbonate, dicalcium phosphate anhydrous, dicalcium phosphate monohydrate, tribasic calcium phosphate, magnesium carbonate, magnesium oxide and the like.

Further, the pellets can be prepared by extrusion and spheronization or roller compaction. The granulate can be dried using a tray drier, fluid bed drier, rotary cone vacuum drier and the like. The dried granulate is then sized. The sizing of granules can be done using an oscillating granulator, comminuting mill or any other conventional equipment equipped with a suitable screen. The dried granulate particles are sieved and then mixed with lubricants and disintegrants and compressed into tablets or pellets or filled into capsules.

Pharmaceutical compositions as disclosed in context of the present invention are used as cholesterol lowering agents

The following examples will further illustrate certain specific aspects and embodiments of the invention in greater detail and are not intended to limit the scope of the invention.

### EXAMPLE 1

### Compositions of Simvastatin Tablets with D₉₀ 10 μm

| | Quantity/Batch (kg) | | | | |
|---|---|---|---|---|---|
| Ingredient | 80 mg | 40 mg | 20 mg | 10 mg | 5 mg |
| Simvastatin^{#} | 6.05 | 3.02 | 1.51 | 0.76 | 0.38 |
| Lactose monohydrate | 42.25 | 21.12 | 10.56 | 5.28 | 2.64 |
| (Impalpable) | | | | | |
| Microcrystalline | 4.5 | 2.25 | 1.12 | 0.56 | 0.28 |
| cellulose | | | | | |
| (Avicel pH 101)* | | | | | |
| Pregelatinised starch | 9.75 | 4.86 | 2.44 | 1.22 | 0.61 |
| Ascorbic acid | 1.5 | 0.75 | 0.375 | 0.19 | 0.09 |
| Citric acid anhydrous | 0.75 | 0.37 | 0.19 | 0.09 | 0.05 |
| Butylated hydroxy | 0.006 | 0.003 | 0.0015 | 0.0008 | 0.0004 |
| anisole | | | | | |
| Water | 7 | 3.5 | 1.75 | 0.87 | 0.44 |
| Isopropyl alcohol | 0.75 | 0.375 | 0.19 | 0.09 | 0.045 |
| Magnesium stearate | 0.9 | 0.45 | 0.22 | 0.11 | 0.05 |

FILM COATING

| | | | | | |
|---|---|---|---|---|---|
| Opadry Brown** | 4.32 | 2.16 | 1.08 | 0.54 | 0.27 |
| Water | 29 | 14.5 | 7.25 | 3.62 | 1.81 |

^{#} Simvastat in D₉₀ = 10 μm
*FMC Biopolymer manufactures Avicel pH 101.
**Opadry Brown is a formulated coating manufactured by Colorcon, West Point, Pennsylvania, U.S.A. and contains hydroxypropyl cellulose, hydroxypropyl methylcellulose, titanium dioxide and iron oxide.

### Manufacturing Process:

1. Simvastatin, ascorbic acid and citric acid anhydrous were sifted through an ASTM # 30 mesh sieve.
2. Lactose monohydrate, microcyrstalline cellulose and about half of the pregelatinized starch were sifted together through an ASTM 30 mesh sieve.
3. Ingredients of step 1 and 2 were sifted together through an ASTM 30 mesh sieve.
4. Materials of step 3 were loaded into a rapid mixer granulator and mixed for 20 minutes.
5. Butylated hydroxy anisole (“BHA”) was dispersed in isopropyl alcohol with stirring for 10 minutes.
6. The dispersion of step 5 was added to water slowly to prevent precipitation of BHA from water, while stirring for 20 minutes.
7. The dispersion of step 6 was used to granulate the dry mix prepared in step 4.
8. The granules of step 7 were dried in a fluid bed drier bowl at an inlet air temperature of 55±5° C. until the loss on drying (“LOD”) was between 1 and 3.5% w/w at 105° C. by infrared moisture analyzer.
9. The dried granules were sifted through an ASTM 20 mesh sieve and the retentions were milled with a 1.5 mm screen at medium speed.
10. Microcrystalline cellulose, the remaining pregelatinized starch and magnesium stearate were sifted through an ASTM 40 mesh sieve.
11. The blends of steps 9 and 10 were loaded into a double cone blender and blended for 5 minutes.
12. The lubricated blend of step 11 was compressed on rotary compression machine.
13. Opadry Brown was dispersed in water with continuous stirring for 45 minutes.
14. After the parameters were set, tablets were warmed while jogging in the coating pan until the tablet bed temperature reached 45±5° C. and the coating was done until the weight gain was 2-3% w/w.
15. After completion of coating, the tablets were dried at 45±5° C. for 15-20 minutes.
   In vitro dissolution profile of simvastatin 80 mg tablets was studied under the following conditions:

Medium: pH 7.0 phosphate buffer containing 0.5% w/v sodium lauryl sulphate.

Apparatus: USP apparatus type 2 (Paddle type) from Test 711 “Dissolution” in United States Pharmacopeia 29, United States Pharmacopeial Convention, Inc., Rockville, Md. (2005).

Liquid volume: 900 ml.

Rotation speed: 50 rpm.

| | Cumulative Drug Release |
|---|---|
| Time (minutes) | (%) |
| 0 | 0 |
| 10 | 89.8 |
| 20 | 98 |
| 30 | 98.7 |
| 45 | 100 |

Three batches of micronized simvastatin, representative of the material used in this example, were characterized using a laser light scattering instrument. The following data were obtained:

| | Particle Size Distribution (μm) | | |
|---|---|---|---|
| Sample | D₁₀ | D₅₀ | D₉₀ |
| A | 1.2 | 3.7 | 8.7 |
| B | 1.2 | 3.7 | 9.2 |
| C | 1.2 | 3.7 | 8.3 |

### EXAMPLE 2 (COMPARATIVE)

Composition of simvastatin 80 mg tablets with D₉₀ 40 μm.

The composition and procedure were similar to that given in Example 1, except for use of simvastatin with D₉₀ of 40 μm and the entire quantity of pregelatinized starch was added extragranularly (i.e., in step 10).

The in vitro dissolution profile of simvastatin 80 mg tablets was studied under the same conditions described in Example 1.

| | Cumulative Drug |
|---|---|
| Time (minutes) | Release (%) |
| 0 | 0 |
| 10 | 77.8 |
| 20 | 95.5 |
| 30 | 97.5 |
| 45 | 96.8 |

### EXAMPLE 3

### Bioavailability Study of Simvastatin 80 mg Tablets with Different Drug Particle Size Distribution

A two-way, two-treatment crossover study design was used to evaluate in vivo performance of simvastatin 80 mg tablets of Example 1 and Example 2 (Comparative) in healthy human volunteers under fasted conditions.

Test/Reference* Ratio

| | | Example 2*** |
|---|---|---|
| Parameter | Example 1** | (Comparative) |
| AUC | 100.6 | 122.4 |
| AUC_{(0–infinity)} | 102.6 | 122.7 |
| Cₘₐₓ | 95.6 | 95.8 |

*Reference was commercially available ZOCOR ™ 80 mg tablets.
**Number of subjects = 70
***Number of subjects = 44

1. A solid pharmaceutical composition prepared using simvastatin particles having a particle size distribution D₉₀ less than about 10 μm.

2. The solid pharmaceutical composition of claim 1, wherein simvastatin particles have a specific surface area between about 4 m²/g and about 7 m²/g.

3. The solid pharmaceutical composition of claim 1, wherein simvastatin particles have a specific surface area between about 4.5 m²/g and about 6.5 m²/g.

4. A process for preparing a solid pharmaceutical composition, comprising combining simvastatin particles having a particle size distribution D₉₀ less than about 10 μm with at least one pharmaceutically acceptable excipient.

5. The process of claim 4, wherein simvastatin particles have a specific surface area between about 4 m²/g and about 7 m²/g.

6. The process of claim 4, wherein simvastatin particles have a specific surface area between about 4.5 m²/g and about 6.5 m²/g.
